(19) United States
(12) Patent Application Publication
(10) Pub. No.: US 2006/0258997 A1
(43) Pub. Date: Nov. 16, 2006

(54) Pouch for ostomy bag

(76) Inventor: Sheila Belt, Rockford, IL (US)

Correspondence Address: WILHELM LAW SERVICE, S.C., 100 W LAWRENCE ST, THIRD FLOOR, APPLETON, WI, 54911, US

(21) Appl. No.: 11/355,487

(22) Filed: Feb. 15, 2006

Related U.S. Application Data
(60) Provisional application No. 60/679,740, filed on May 11, 2005.

(51) Int. Cl. A61F 5/44

(52) U.S. Cl. 604/345; 604/339

(57) ABSTRACT

Pouches which receive and contain ostomy bags which are in use by ostomy patients. The body-facing wall of the pouch covers substantially the entirety of the facing surface of the ostomy bag, except for portions of the bag which are necessarily exposed to the user's body in attaching the ostomy bag to the stoma. The body-contacting surface of the body-facing wall of the pouch is made of material which is skin-friendly, typically cotton, optionally 100 percent soft absorbent cotton; and is optionally sewn with cotton thread. The cotton in the body-contacting layer is typically uncolored, optionally unbleached. An opening in the pouch is used to insert and remove ostomy bags, into and out of the pouch. An aperture in the pouch can be expanded for insertion of the ostomy bag into the pouch, and for removal of the ostomy bag from the pouch.

### BACKGROUND

This invention relates generally to use of ostomy bags by, for example, people who have had a portion of their colon removed such that the solid body waste, namely feces, exits the person's body through a portion of the intestine which extends through the front portion of the abdomen, rather than exiting the body at the anus.

In such situations, an appliance is adhesively bonded to the abdomen and is releasably mounted to the intestine. The appliance is connected, disconnected, and reconnected as needed, to disposable plastic ostomy bags which receive the body waste material.

Such people are constantly wearing a plastic ostomy bag, which receives excretions on an involuntary basis. Namely, the person has no control over when or how much waste material is excreted by the body. Thus, the plastic ostomy bag is worn at all times. This plastic bag is thus at all times in surface-to-surface contact with the soft skin at the front of the abdomen.

Wearers of such ostomy bags typically suffer from various skin problems associated with the constant contact between the plastic ostomy bag and the skin. Such skin problems are associated with the rubbing, abrasion, friction, and the like between the plastic and the skin, as well as the fact that moisture and other materials released by the skin become trapped between the skin and the bag. In general, the plastic bag does not allow the underlying skin to “breathe”, does not allow the trapped materials to evaporate from the skin. Foreign material such as dirt and the like are captured by the moisture and can further irritate the skin. The skin site is thus constantly wet or damp and quickly becomes unsanitary, whereby the skin is exposed to disease-causing materials over prolonged periods of time. As a result, it is common for the skin proximate a stoma to exhibit one or more moisture-related skin diseases or infections, including bacteria, mold, and yeast growth on the skin.

Wearers of ostomy bags typically try to conceal the presence of the bag so as to not attract attention to their use of such bags. It is important to the stoma user that any stoma-related product be consistent with such concealment.

The problem addressed by the invention is that of providing structures and processes which assist in attenuating development of skin problems associated with use of plastic ostomy bags. The problem is solved by a novel pouch which, among other features, is discrete in design, thus generally invisible under the wearer's regular clothing. The pouch receives and carries, and readily accommodates replacement of, a conventional plastic ostomy bag. The pouch is made of a skin-friendly material which spaces the plastic ostomy bag from the wearer's skin, and facilitates evaporation of moisture from the skin, so as to maintain the skin in a relatively dryer condition. With the moisture level at the surface of the skin thus reduced, the skin surface does not hold dirt and other detritus as readily whereby the skin is cleaner. In addition, the dryer environment of the skin surface is an unfriendly environment for growth of bacteria, mold, yeast, and the like. The overall result is a substantial reduction in populations of disease-causing organisms on the skin surface, and a resultant reduction in skin diseases at and proximate the site of the stoma.

### SUMMARY

The invention provides a flexible pouch which receives and contains an ostomy bag which is being used by an ostomy patient. The body-facing wall of the pouch covers substantially the entirety of the facing surface of the ostomy bag, except for portions of the bag which are necessarily exposed to the user's body in attaching the ostomy bag to the stoma. A connector on the ostomy bag extends through an aperture in the body-facing wall of the pouch, and connects to the stoma. The aperture can be expanded so as to enable an ostomy bag to be inserted into the pouch cavity, and to be removed from the pouch when a suitable quantity of material has been collected in the bag. The body-contacting surface of the body-facing wall of the pouch is made of material which is skin-friendly, typically cotton, optionally 100 percent soft absorbent cotton; and is optionally sewn with cotton thread. The cotton in the body-contacting layer is typically uncolored, optionally unbleached. An opening in the pouch is used to insert and remove ostomy bags, into and out of the pouch. Structure

In a first family of embodiments, the invention comprehends a pouch having a length and a width. The pouch is adapted and configured to receive an ostomy bag. The ostomy bag has a body-facing side and a front-facing side. The body-facing side has an opening adapted and configured to receive a stoma. The pouch has an upper portion and a lower portion, and comprises a body-facing wall having a first upper wall portion defined in the upper portion of the pouch, a first lower wall portion defined in the lower portion of said pouch, and first and second opposing side edges; a front-facing wall having a second upper wall portion defined in the upper portion of the pouch, a second lower wall portion defined in the lower portion of the pouch, the front-facing wall being joined to the body-facing wall so as to define a cavity in the pouch between the lower portions of the body-facing wall and the front-facing wall; an aperture extending through the body-facing wall, disposed generally between the upper and lower portions of body-facing wall and providing ingress into, and egress out of, the cavity; and one or more breaks in at least one of the body-facing wall and the front-facing wall, extending from the aperture toward the opposing side edges of the body-facing wall, so as to enable expanding the aperture (16) to define an enlarged opening which provides access to the cavity between the body-facing wall and the front-facing wall, the opening being adapted and configured to receive an ostomy bag therethrough and into the cavity.

In some embodiments, the opening extends from the first side edge along a first one of the breaks, across the aperture, and along a second one of the breaks.

In some embodiments, the upper portion of the pouch is adapted and configured to accommodate inverting the upper portion of the body-facing wall about the front-facing wall thereby to define the opening.

In some embodiments, the upper portion of the pouch is adapted and configured to accommodate folding the outer surface of the upper portion of the front-facing wall back onto the outer surface of the lower portion of the front-facing wall, and carrying the upper portion of the body-facing wall with the upper portion of the front-facing wall, thereby to define the opening.

In some embodiments, when the pouch is folded back, with the upper portion of the front-facing wall facing the lower portion of the front-facing wall, one or more tabs on at least one of the upper portion of the body-facing wall and the lower portion of the body-facing wall extends substantially above a lower edge the opening.

In some embodiments, a body-facing layer of the body-facing wall is an unconsolidated layer of a skin-friendly material such as 100 percent cotton, for example unbleached and/or uncolored cotton.

In some embodiments, the body-facing wall comprises a multiple layer structure wherein a body-facing layer of the multiple layer structure is defined by the skin-friendly material.

In a second family of embodiments, the invention comprehends a pouch which is adapted and configured to receive an ostomy bag therein, wherein the ostomy bag has a body-facing side and a front facing side, the pouch having an upper portion and a lower portion, and comprising a body-facing wall, having first and second opposing lateral side edges; a front-facing wall, joined to the body-facing wall so as to define a cavity in the pouch between the body-facing wall and the front-facing wall; an access opening in the body-facing wall, which is adapted and configured to be temporarily extended, in a generally consistent direction, from the first lateral side edge in a generally transverse direction across the body-facing wall to an aperture, across the aperture, and from the aperture to the second lateral side edge, the access opening providing ingress into, and egress out of, the cavity.

In some embodiments, the body-contacting layer is supported by a substrate layer, the substrate layer having a composition different from the skin-friendly material of the body-contacting layer.

In a third family of embodiments, the invention comprehends a pouch adapted and configured to receive an ostomy bag therein, wherein the ostomy bag has a body-facing side and a front-facing side, and wherein the body-facing side has a structure adapted to receive a stoma, the pouch having an upper portion and a lower portion, and comprises a body-facing wall; a front-facing wall; a cavity in the pouch between the body-facing wall and the front-facing wall; and an access opening in the body-facing wall, providing ingress into, and egress out of, the cavity, the body-facing wall comprising a body-contacting layer which is fabricated from a skin-friendly material. The body-facing wall is optionally joined to the front-facing wall using a skin-friendly thread, whereby the entirety of that portion of the pouch which interfaces with a user's body is made from skin-friendly material, such as cotton, optionally uncolored cotton and/or unbleached cotton.

In some embodiments the upper portion of the pouch is adapted and configured to accommodate inverting the upper portion of the body-facing wall about the front-facing wall thereby to define an enlarged opening.

In a fourth family of embodiments, the invention comprehends a method of preventing deleterious response of a user's skin to an ostomy bag, wherein the ostomy bag comprises a plastic receptacle, the method comprising generally enclosing the plastic receptacle in a pouch, the pouch having a body-facing wall, a body-contacting layer of the body-facing wall being made from skin-friendly material, whereby the entirety of the plastic receptacle is spaced from the user's skin by the skin-friendly material.

In a fifth family of embodiments, the invention comprehends a method of loading an ostomy bag into an ostomy bag pouch, comprising displacing an upper portion of the pouch from a lower portion of the pouch thus to define an opening associated with a top of the lower portion of the pouch, inserting the ostomy bag into the pouch through the opening, connecting the ostomy bag to the stoma; closing the pouch about the ostomy bag so as to limit contact, between the ostomy bag and skin of a wearer, to skin which is immediately proximate the stoma and skin which is immediately proximate any flange on the ostomy bag and which is proximate such stoma when the ostomy bag is connected to the stoma.

### BRIEF DESCRIPTION OF THE DRAWINGS

FIG. 1 shows a pictorial view of an ostomy bag pouch of the invention.

FIG. 2 shows an elevation view of the body-facing side of the pouch of FIG. 1.

FIG. 3 shows an elevation view of the front-facing side of the pouch of FIG. 1.

FIG. 4 shows a cross-section representation of the pouch of FIG. 2 and is taken at 4-4 of FIG. 2.

FIG. 5 shows a side view representation of the pouch of FIG. 1 with the upper portion of the pouch folded inside out, and showing an ostomy bag received in the pouch.

FIGS. 5A, 5B, and 5C illustrate a second embodiment of pouches of the invention.

FIG. 6 shows a cross-section representation of the pouch and ostomy bag of FIG. 5 with the upper portion of the pouch returned to the right-side-out, closed configuration.

FIG. 7 shows a reduced-size perspective view of the pouch of FIG. 1 being worn by a user thereof.

The invention is not limited in its application to the details of construction or the arrangement of the components set forth in the following description or illustrated in the drawings. The invention is capable of other embodiments or of being practiced or carried out in various other ways. Also, it is to be understood that the terminology and phraseology employed herein is for purpose of description and illustration and should not be regarded as limiting. Like reference numerals are used to indicate like components.

### DESCRIPTION OF THE ILLUSTRATED EMBODIMENTS

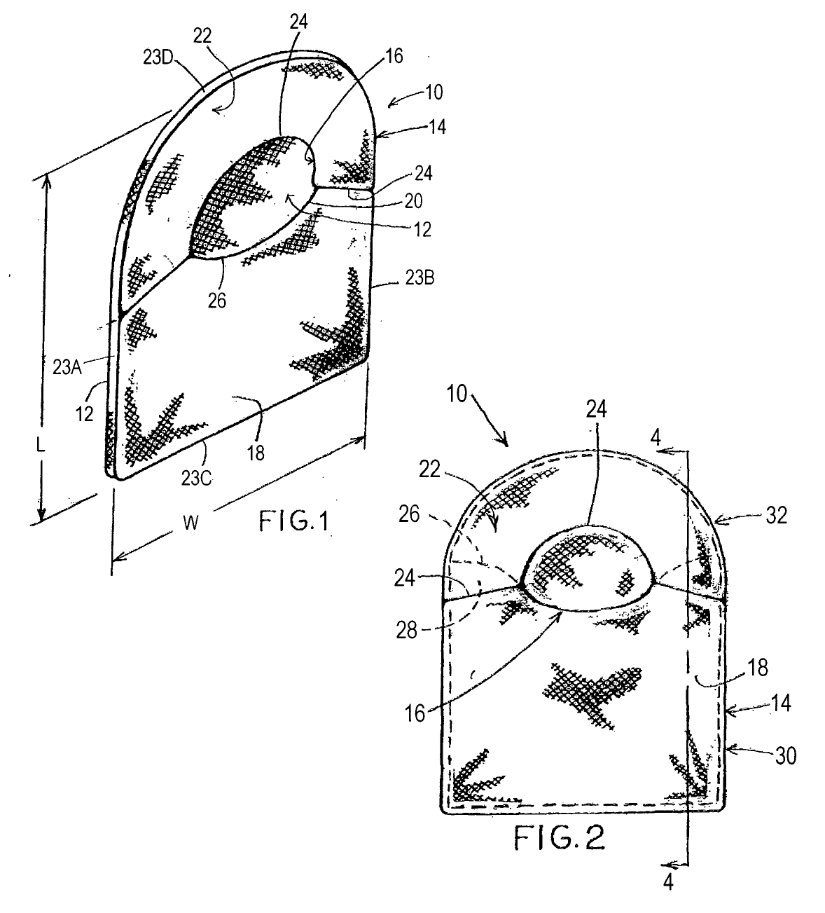

The general outline of the ostomy bag-receiving pouch of the invention is shown in FIGS. 1-3, and 7. Pouch 10 has a length “L” and a width “W”, a front-facing wall 12 and a body-facing wall 14. An aperture 16 extends through body-facing wall 14 into the interior of the pouch. Body-facing wall 14 has a lower panel 18 which extends down from generally the lower edge 20 of aperture 16, and an upper panel 22. Left and right side edges 23A, 23B represent the side edge extremities of both body-facing wall 14 and front-facing wall 12. Bottom edge 23C represents the bottom edge extremities of both body-facing wall 14 and front-facing wall 12. Arcuate top edge 23D represents the top edge extremities of both body facing wall 14 and front-facing wall 12.

Referring to FIGS. 1 and 2, the lines 24 which extend outwardly and at a down-sloping angle, on opposing sides of aperture 16, generally represent lower edges of upper panel 22. Dashed lines 26 represent the upper edges of lower panel 18 of body-facing wall 14. Upper edges 26 underlie lower edges 24, thus to define upwardly-extending tabs 28 which underlie the lower portions of upper panel 22, and extend above the lower edge of aperture 16. Upper edges 26 and tabs 28 are disposed inwardly in the pouch relative to panel 22 at lower edges 24. Accordingly, upper panel 22 and lower panel 18 of the body-facing wall of the pouch are generally separate and distinct elements, not joined to each other, except through their collective joinder to front-facing wall 12 e.g. at the seams by which front-facing wall 12 is joined to body-facing wall 14, especially where tabs 28 overlap with the lower portion of upper panel 22 of the body-facing wall. The upper (26) and lower (24) edges thus represent overlapping discontinuities in moving along a path starting at the bottom of wall 14 and progressing to the top of wall 14.

Pouch 10 can be defined as having a lower portion 30 and an upper portion 32. Lower portion 30 is defined generally below the lower edge of aperture 16, in both body-facing wall 14 and front-facing wall 12. Upper portion 32 is defined generally as that portion of the pouch, in both body-facing wall 14 and front-facing wall 12, which is above the lower edge of aperture 16 in the orientations shown in FIGS. 1-3.

In the embodiments seen in e.g. FIGS. 1-3, the upper edge of upper portion 32 is curvilinear, and is illustrated as being circular. A variety of shapes are contemplated for the outer edges of pouch 10. A shape which generally complements the outline of the ostomy bag to be received therein is generally quite satisfactory for the purpose intended.

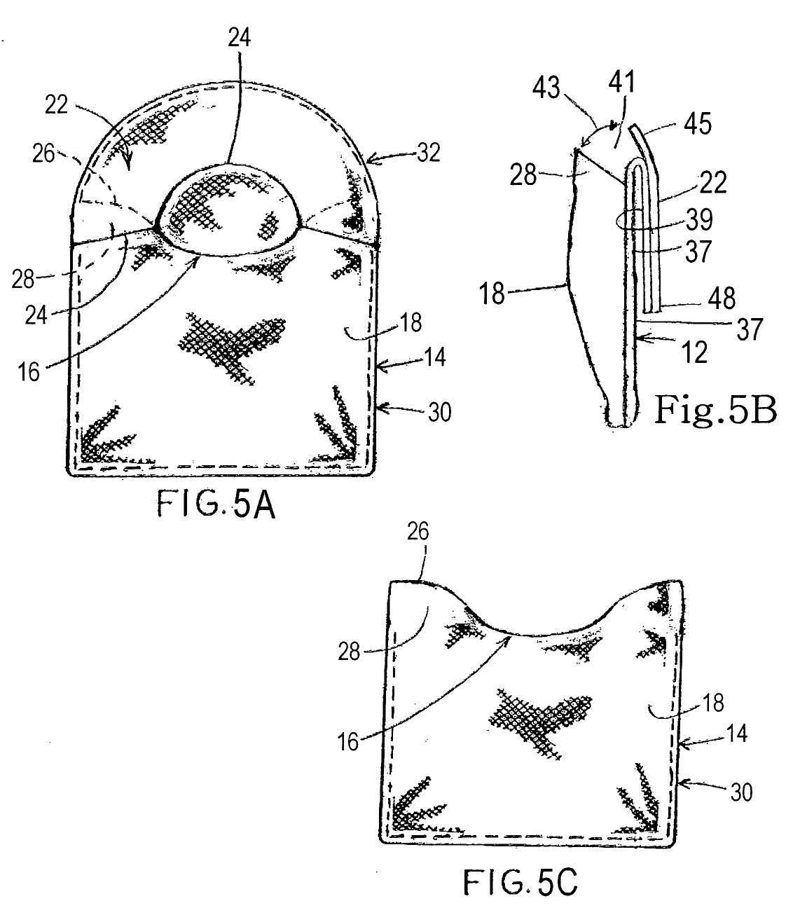

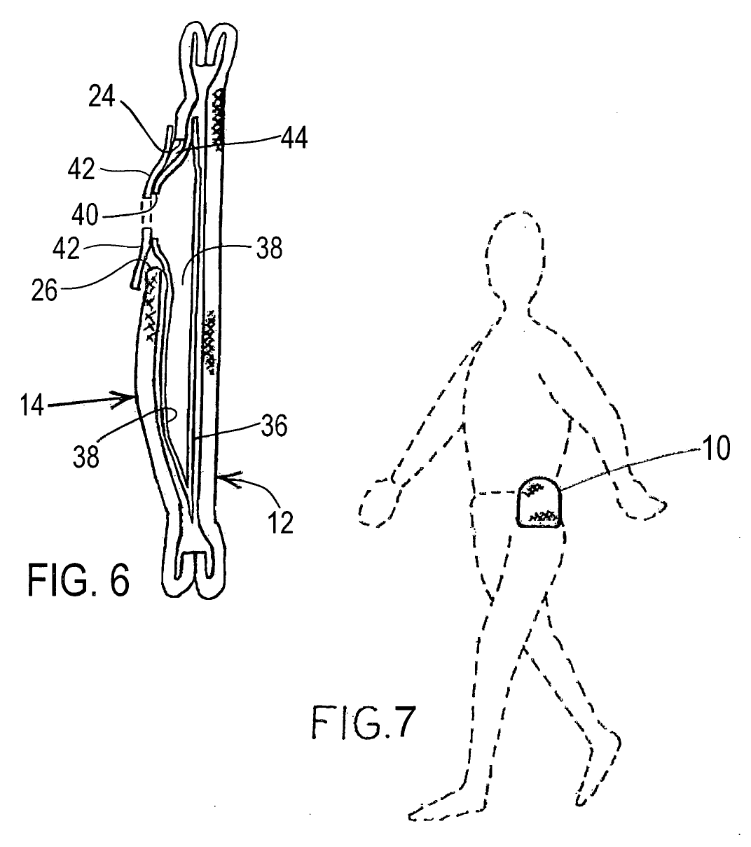

Pouch 10 is sized and configured such that plastic ostomy bags 34 of one or more conventionally-available sizes and configurations are readily received in a cavity 36 (FIG. 6) in e.g. lower portion 12 of the pouch. A typical such ostomy bag includes a plastic receptacle 38 (FIG. 6), which has a waste-receiving opening 40. Receptacle 38 receives and stores the body waste. Bag 34 can include a flange 42 attached to the bag about opening 40. The flange extends away from the opening, thus to define an annular recess 44 (FIG. 5) about opening 40 and between flange 42 and the body of bag 34. The length and width of pouch 10 generally corresponds with a size and configuration of certain conventionally available ostomy bags. Thus, the general length “L” and width “W” of the pouch closely tracks the length and width of a readily available size and configuration of ostomy bag, and is modestly larger than the size and configuration of the respective conventionally available ostomy bags.

An opening 41 across the width of pouch 10 can be defined adjacent aperture 16 where upper edge 26 of lower panel 18 and the lower part of upper panel 22 face each other in generally overlapping relationship. The overlap, in the relaxed, unfolded, condition of the pouch (FIGS. 2, 4) generally closes the pouch to ingress and egress of the ostomy bag, or other material, thereby to hold and retain the ostomy bag in the pouch.

In consideration of the configurations of tabs 28, considering that the upper 22 and lower 18 panels of the body-facing wall are generally not attached to each other across width “W” of the pouch, considering that the pouch material is a very flexible woven fabric, the upper portion of the pouch can be folded inside out, about the sewn seam 48 at threads 50, to an inverted-top configuration. After such folding, seam 48, which is inside the pouch cavity in FIG. 4, is part of the outer surface of the upper portion of the pouch. Similarly, the surfaces 46 of the body-facing wall and the front-facing wall, which define inner surfaces of the pouch in FIG. 4, form part of the outer surface of the upper portion of the pouch in the opened-pouch configuration illustrated in FIG. 5. Such folding displaces the top panel of the body-facing wall from the lower panel of the body-facing wall and thus generally opens the pouch at the overlap area, adjacent tabs 28, at the area of overlap of the upper and lower portions of the body-facing sidewall. With the pouch open, the wearer or a care-giver has unobstructed access, through opening 41, to cavity 36, and to an ostomy bag 34 received in the cavity. The wearer or care-giver can thus inspect the ostomy bag and its content, and can readily remove and/or replace the ostomy bag as desired.

In an alternate set of embodiments of pouch 10, illustrated in FIGS. 5A, 5B, and 5C, stitching 50 is discontinued across tabs 28 such that the underlying tabs 28 and the lower edges of upper panel 22, are not secured to front-facing wall 12. In creating opening 41, the user folds the outer surface of the upper portion 39 of front-facing wall 12 back against the outer surface of the lower portion 37 of front-facing wall 12, as illustrated in FIG. 5B. Such folding of the upper portion of the front-facing wall carries along the upper panel 22 of the body-facing wall, thereby to create a temporary opening 41 in the body-facing wall, at the top of lower panel 18 of the body-facing wall, and to expose tabs 28.

FIG. 5C shows the folded-back pouch of FIG. 5B looking in a direction toward the body-facing panel. Referring to both FIGS. 5B and 5C, tabs 28 and the lower portion of panel 22 are not attached to front-facing wall 12 or to each other whereby, in the folded orientation of FIGS. 5B and 5C, the lower portion of panel 22 defines a second set of tabs 45. In the open configuration illustrated in FIGS. 5B and 5C, tabs 28 and 45 extend upwardly on opposing sides of opening 41. Opening 41 extends generally across the entire width of pouch 10, from side edge 23A to aperture 16, across the lower edge of aperture 16, and from aperture 16 to side edge 23B. The full extent of the width of opening 41, across the entirety of the width of the pouch, along with the upward framing of opening 41 by tabs 28 and 45, assists in guiding insertion of an ostomy bag into the pouch.

A used ostomy bag in the pouch, e.g. an ostomy bag which is sufficiently full that the bag should be removed from the pouch, is readily removed from receptacle 38 either when the upper panel of the body-facing wall is inverted about the upper portion of the front-facing wall as illustrated in FIG. 5, or when the pouch is so folded as illustrated in FIGS. 5B and 5C. The pouch is removed by displacing the upper panel of the body-facing wall so as to expose opening 41, disconnecting the bag from the stoma, and pulling the bag out of the pouch. The pouch, still configured with opening 41 exposed, is ready to receive a new, clean ostomy bag. The bag is inserted through opening 41, whereby the lower portion of the body-facing wall is positioned between the lower portion of the plastic ostomy bag and the user's skin. The upper panel of the body-facing wall is then drawn and reconfigured about the upper portion of the newly-inserted ostomy bag thus to bring the skin-friendly upper panel of the body-facing wall into position between the upper portion of the ostomy bag and the user's skin.

Once the clean ostomy bag has been properly installed in the pouch, the top portion of the pouch is restored, from the inverted-top configuration of FIG. 5, to the closed-top configuration illustrated in FIG. 6. A portion of upper panel 22 of the body-facing wall, e.g. above aperture 16, may, in some embodiments, be tucked into recess 44, between flange 42 and the receptacle portion 38 of the bag, as seen in FIG. 6. This closes the pouch about the ostomy bag receptacle, whereby the upper 22 and lower 18 panels are returned to overlapping relationship with each other at tabs 28, which returns the appearance of the pouch to that of the discrete, slim configuration shown in FIGS. 1-4.

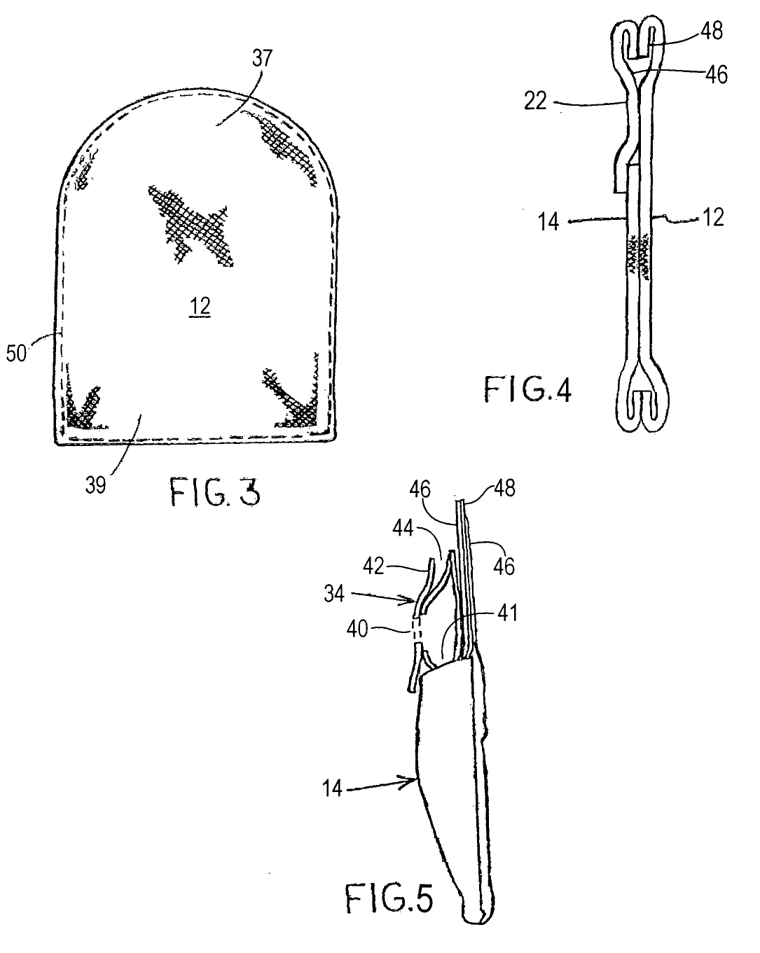

The outer surface of front-facing wall 12 of the pouch can be undecorated, and plainly-configured, as illustrated in FIG. 3, and need not have any structural elements or graphics thereon, thereby to provide a surface which is readily concealed in order to avoid revealing the presence of the ostomy bag 34 or pouch 10 under the wearer's outer clothing.

In general, pouch 10 is sized and configured such that conventionally-available ostomy bags, by size and configuration, are received in the lower portion of the pouch, and the entrance to the bag extends through aperture 16 as shown in FIG. 6, so as to be accessible for connection to the stoma which exits the wearers abdomen.

By receiving and holding the ostomy bag in a skin-friendly pouch, such that the skin is not exposed to regular and extended contact with the plastic of the ostomy bag, other than at flange 42, moisture level at the surface of the skin is substantially reduced such that skin problems which commonly attend use of ostomy bags are substantially avoided, and in some cases are completely, avoided.

The material seen through the aperture, in FIGS. 1 and 2, is part of the inner surface of front-facing wall 12 of the pouch.

A critical feature of pouch 10 is that the materials of which the body-facing wall is made must be skin friendly. Namely, the material should function so as to promote skin health, or at least not negatively affect skin health. Preferred properties of such material is that it has no negative interaction with the skin; nor does it provide a hospitable environment for residence by material which does have a negative interaction with, or affect on, skin. There should be no deleterious physical stress, no chemical stress, no mechanical stress, on the skin as a result of such material being in close proximity with, or contact with, the skin for prolonged periods of time. Nor should such material create such conditions as moisture entrapment which provides a hospitable environment for growth of organisms which successfully attack the skin.

The above stated performance conditions screen out a large fraction of the known sheet materials which are susceptible to being made into pouch-like structures. Of those materials which do meet the above-stated performance conditions, the material which has been found most friendly to the skin is 100 percent cotton. An unbleached, unprinted cotton is highly desirable because it has not been subjected to the chemicals which accompany bleaching processes and printing processes, and thus has no residual content of such chemicals.

Accordingly, pouch 10 is typically made of all cotton material so as to be skin-friendly. The stitching 50 (FIG. 2) at seams 48 (FIGS. 4 and 5) is also typically all cotton for the same reason. In some instances, the stitching need not be cotton, especially where the skin of the wearer is quite robust, and not especially sensitive to e.g. polyester or other thread material in small quantities. However, for mass production of pouches 10 to be used by a broad spectrum of the human population, which is not screened other than as to need for avoiding skin problems associated with plastic ostomy bags, a highly preferred material for use as the body-facing wall is cotton, both the woven fabric and the thread.

In some embodiments, the joinder of the front-facing wall and the body-facing wall can be effected by other than stitching. There can be mentioned, for example and without limitation, adhesive bonding, and ultrasonic bonding without use of chemical adhesive.

In some embodiments, the body-facing wall can be made of material which embodies non-cotton fibers such as polyester, in limited amounts typically up to less than 20 percent by weight of the fiber, optionally less than 10 percent by weight. However, the most preferred embodiments comprehend 100 percent cotton in the body-facing wall.

Use of 100 percent cotton in the body-facing wall provides a pouch which is acceptable to the skin of the largest population of potential users. Where the cotton used for the body-facing wall is uncolored, e.g. not printed, any potential for skin reaction to dyes, pigments, or the like is avoided. Where the cotton used for the body-facing wall is unbleached, potential for skin reaction to materials left in the cotton, by the bleaching process, is avoided. Accordingly, unbleached, optionally uncolored, cotton is an especially desirable, though not limiting, material for use as the body-facing wall.

As an option, body-facing wall 14 can be fabricated using multiple layers of material, or a previously-constructed sheet material. The multiple layers can be attached to each other in intimate surface-to-surface contact, or can be loosely attached to each other, or can be generally unattached to each other except at the edge of the respective panels 18, 22. In some instances, a first material layer can be attached to a second layer without any consolidation or individual integrity of the first layer. Thus, the outer layer can be consolidated or unconsolidated. For example, a flocked layer can be deposited on a substrate layer.

In such multiple layer structures, it is necessary that the surface layer which is to be in contact with the user's skin be a skin-friendly material such as cotton. The other layers can be selected for other properties and functions such as strength, cost, dimensional stability, and the like, so long as they accommodate the skin-friendly features of the skin-contacting layer.

Thus, panels 18, 22 can be constructed from multiple layer materials wherein e.g. a light-weight cotton surface layer, or simply cotton fibers, are mounted on a substrate layer. The cotton layer is disposed on the outer surface of pouch 10.

The substrate layer can be a single layer or multiple layers. As material for the substrate layer, there can be mentioned, for example and without limitation, woven or non-woven sheet materials, as well as a variety of polymeric sheet materials, all of which have been fabricated so as to support achievement of a relatively dry skin at the skin-cotton interface. As the non-woven and woven sheet materials, there can be mentioned a wide variety of natural and synthetic fibers including, without limitation, cotton, wool, linen, silk, polyester, nylon, rayon, acrylics. As polymeric sheet materials, there can be mentioned a wide variety of e.g. extruded films such as polyethylenes, polypropylenes, vinyls, acetates, acrylates, and the like. Such materials are known to be treated, such as for use in personal hygiene products, so as to enable passage of moisture vapor, so as to accommodate evaporation of moisture from the skin. All such materials are acceptable so long as they support the skin-friendly properties of the skin-contact layer. Various polymeric materials can also be extruded to form fibers which are converted to sheet form and such sheets are then used in fabricating the substrate layer.

Those skilled in the art will now see that certain modifications can be made to the apparatus and methods herein disclosed with respect to the illustrated embodiments, without departing from the spirit of the instant invention. And while the invention has been described above with respect to the preferred embodiments, it will be understood that the invention is adapted to numerous rearrangements, modifications, and alterations, and all such arrangements, modifications, and alterations are intended to be within the scope of the appended claims.

To the extent the following claims use means plus function language, it is not meant to include there, or in the instant specification, anything not structurally equivalent to what is shown in the embodiments disclosed in the specification.

Having thus described the invention, what is claimed is:

1. A pouch having a length and a width, said pouch being adapted and configured to receive an ostomy bag therein, wherein such ostomy bag has a body-facing side, and a front-facing side and wherein such body-facing side has an opening adapted and configured to receive a stoma, said pouch having an upper portion (32) and a lower portion (30), and comprising:
(a) a body-facing wall (14) having a first upper wall portion defined in the upper portion (32) of said pouch, a first lower wall portion defined in the lower portion (30) of said pouch, and first and second opposing side edges (23A, 23B);
(b) a front-facing wall (12) having a second upper wall portion defined in the upper portion (32) of said pouch, a second lower wall portion defined in the lower portion (30) of said pouch, said front-facing wall being joined to said body-facing wall so as to define a cavity (36) in said pouch between the lower portions of said body-facing wall and said front-facing wall;
(c) an aperture (16) extending through said body-facing wall (14), disposed generally between the upper and lower portions of said body-facing wall and providing ingress into, and egress out of, the cavity (36); and
(d) one or more breaks in at least one of said body-facing wall (14) and said front-facing wall (12), extending from the aperture (16) so as to enable expanding the aperture (16) to define an enlarged opening (41) which provides access to the cavity (36) between the body-facing wall and the front-facing wall, the opening being adapted and configured to receive an ostomy bag therethrough and into the cavity.

2. A pouch as in claim 1 wherein the one or more breaks in the at least one of said body-facing wall and said front-facing wall extend from the aperture toward the opposing side edges (23A, 23B) of the body-facing wall, and enable expanding the aperture (16) by generally displacing the upper portion of said body-facing wall from the lower portion of said body-facing wall, thus to define the enlarged opening (41).

3. A pouch as in claim 1 wherein the opening extends from the first side edge (23A) along a first one (26) of the breaks, across the aperture (16), and along a second one (26) of the breaks.

4. A pouch as in claim 1 wherein the upper portion of said pouch is adapted and configured to accommodate inverting the upper portion of the body-facing wall about the front-facing wall thereby to define the opening.

5. A pouch as in claim 1 wherein the upper portion of said pouch is adapted and configured to accommodate folding the outer surface of the upper portion of said front-facing wall back onto the outer surface of the lower portion of said front-facing wall, and carrying the upper portion of said body-facing wall with the upper portion of said front-facing wall, thereby to define the opening.

6. A pouch as in claim 5 wherein, when said pouch is so folded back, with the upper portion of said front-facing wall facing the lower portion of said front-facing wall, one or more tabs on at least one of the upper portion of said body-facing wall and the lower portion of said body-facing wall extends substantially above a lower edge the opening.

7. A pouch as in claim 1 wherein a body-facing layer of said body-facing wall is an unconsolidated layer of a skin-friendly material.

8. A pouch as in claim 7 wherein said skin-friendly material is 100 percent cotton.

9. A pouch as in claim 1 wherein said body-facing wall comprises a multiple layer structure wherein a body-facing layer of said multiple layer structure is defined by a skin-friendly material.

10. A pouch as in claim 9 wherein said skin-friendly material is 100 percent cotton.

11. In combination, a pouch as in claim 1, and an ostomy bag disposed in the cavity (36) of said pouch.

12. A pouch adapted and configured to receive an ostomy bag therein, wherein such ostomy bag has a body-facing side and a front facing side, said pouch having an upper portion (32) and a lower portion (30), and comprising:
(a) a body-facing wall (14), having first and second opposing lateral side edges (23A, 23B);
(b) a front-facing wall (12), joined to said body-facing wall so as to define a cavity (36) in said pouch between said body-facing wall and said front-facing wall;
(c) an access opening in said body-facing wall, which is adapted and configured to be temporarily extended, in a generally consistent direction, from the first lateral side edge (23A) in a generally transverse direction across said body-facing wall to an aperture (16), across the aperture, and from the aperture to the second lateral side edge (23B), said access opening providing ingress into, and egress out of, the cavity (36).

13. A pouch as in claim 12, said body-facing wall (14) comprising a body-contacting layer, including about and above the aperture (16), which is fabricated from skin-friendly material.

14. A pouch as in claim 12 wherein said body-contacting layer is substantially all cotton.

15. A pouch as in claim 13 wherein said body-contacting layer is all uncolored cotton.

16. A pouch as in claim 13 wherein said body-contacting layer is all unbleached and uncolored cotton.

17. A pouch as in claim 13 wherein said body-contacting layer is supported by a substrate layer, said substrate layer having a composition different from the skin-friendly material of said body-contacting layer.

18. A pouch as in claim 17 wherein said body-contacting layer is substantially all cotton.

19. In combination, a pouch as in claim 12, and an ostomy bag disposed in the cavity (36) of said pouch.

20. A pouch adapted and configured to receive an ostomy bag therein, wherein such ostomy bag has a body-facing side and a front-facing side, and wherein such body-facing side has a structure adapted to receive a stoma, said pouch having an upper portion (32) and a lower portion (30), and comprising:
(a) a body-facing wall (14);
(b) a front-facing wall (12);
(c) a cavity (36) in said pouch between said body-facing wall (14) and said front-facing wall (12); and
(d) an access opening (16) in said body-facing wall, providing ingress into, and egress out of, the cavity (36),
said body-facing wall comprising a body-contacting layer which is fabricated from a skin-friendly material.

21. A pouch as in claim 20, said body-facing wall being joined to said front-facing wall using a skin-friendly thread, whereby the entirety of that portion of said pouch which interfaces with a user's body is made from skin-friendly material.

22. A pouch as in claim 20 wherein said body-contacting layer is cotton.

23. A pouch as in claim 20 wherein said body-contacting layer is uncolored cotton.

24. A pouch as in claim 20 wherein said body-contacting layer is unbleached and uncolored cotton.

25. A pouch as in claim 20 wherein the upper portion of said pouch is adapted and configured to accommodate inverting the upper portion of the body-facing wall about the front-facing wall thereby to define an enlarged opening (41).

26. A pouch as in claim 20 wherein the upper portion of said pouch is adapted and configured to accommodate folding the outer surface of the upper portion of said front-facing wall back onto the outer surface of the lower portion of said front-facing wall, and carrying the upper portion of said body-facing wall with the upper portion of said front-facing wall, thereby to define an enlarged opening (41).

27. A pouch as in claim 26 wherein, when said pouch is so folded back, with the upper portion of said front-facing wall facing the lower portion of said front-facing wall, one or more tabs on at least one of the upper portion of said body-facing wall and the lower portion of said body-facing wall extends substantially above a lower edge the opening.

28. A pouch as in claim 20 wherein said body-contacting layer is an unconsolidated layer.

29. A pouch as in claim 28 wherein said body-contacting layer is 100 percent cotton.

30. In combination, a pouch as in claim 20, and an ostomy bag disposed in the cavity (36) of said pouch.

31. A method of preventing deleterious response of a user's skin to an ostomy bag, wherein such ostomy bag comprises a plastic receptacle, the method comprising generally enclosing such plastic receptacle in a pouch, the pouch having a body-facing wall, a body-contacting layer of said body-facing wall being made from skin-friendly material, whereby the entirety of the plastic receptacle is spaced from the user's skin by the skin-friendly material.

32. A method as in claim 31 wherein thread used in assembling the pouch is made with skin-friendly material.

33. A method as in claim 31 wherein the body-contacting layer is all cotton.

34. A method of loading an ostomy bag into an ostomy bag pouch, comprising:
(a) displacing an upper portion of a wall of the pouch from a lower portion of the respective wall of the pouch thus to define a generally laterally-extending opening associated with a top of the lower portion of the pouch, the opening extending generally across the width of the pouch;
(b) inserting the ostomy bag into the pouch through the opening, connecting the ostomy bag to the stoma;
(c) closing the pouch about the ostomy bag so as to limit contact, between the ostomy bag and skin of a wearer, to
(i) skin which is immediately proximate the stoma and
(ii) skin which is immediately proximate any flange on the ostomy bag and which is proximate such stoma when the ostomy bag is connected to the stoma.
